(19) United States
(12) Patent Application Publication
(10) Pub. No.: US 2010/0178266 A1
(43) Pub. Date: Jul. 15, 2010

(54) Process For The Manufacture Of Polyorganosiloxanes Comprising (C6-C60)-Alkylmethylsiloxy Groups And Dimethylsiloxy Groups

(75) Inventors: John Huggins, Leverkusen (DE); Martin Kropfgans, Odenthal (DE); Gunnar Hoffmueller, Leverkusen (DE); Hubertus Eversheim, Wermelskirchen (DE)

Correspondence Address: RANKIN, HILL & CLARK LLP, 23755 Lorain Road - Suite 200, North Olmsted, OH, 44070-2224, US

(73) Assignee: MOMENTIVE PERFORMANCE MATERIALS GMBH, Leverkusen (DE)

(21) Appl. No.: 12/667,304

(22) PCT Filed: Jun. 23, 2008
(86) PCT No.: PCT/EP08/57945

§ 371 Date: Dec. 30, 2009

(30) Foreign Application Priority Data
Jul. 2, 2007 (DE) 102007030642.5

(51) Int. Cl. A61K 8/891; A61Q 5/12; A61K 31/765; A61Q 19/00; C08G 77/08; C09K 3/22

(52) U.S. Cl. 424/70.12; 424/78.03; 528/32; 252/88.1

(57) ABSTRACT

The present invention relates to novel processes for the manufacture of poly-organosiloxanes comprising (C6-C60)-alkylmethylsiloxy-groups and dimethyl-siloxy groups, polyorganosiloxanes obtainable by such processes, preferably aqueous emulsions comprising such polyorganosiloxanes and the use of such polyorganosiloxanes.

The present invention relates to novel processes for the manufacture of poly-organosiloxanes comprising (C6-C60)-alkylmethylsiloxy-groups and dimethyl-siloxy groups, polyorganosiloxanes obtainable by such processes, aqueous emulsions comprising such polyorganosiloxanes and the use of such polyorganosiloxanes. The polyorganosiloxanes prepared by the process of this invention are particularly useful in and/or as lubricating compositions, leather care compositions, polishing compositions, especially car polishing, foam stabilizers, anti-foam agents, rheological additives, for example in paint compositions, release agents, in particular, mold release agents, high refractive index additives, and personal care compositions, like cosmetic formulations.

### TECHNICAL PROBLEM

Poly(C6-C60)-alkylmethylsiloxanes are useful in personal care formulations for skin creams and hair care shampoos and conditioners (see e.g. U.S. Pat. No. 5,578,692, WO 91/009586). Among all the different possibilities of structural modifications of the poly(C6-C60)-alkylmethylsiloxanes i.e. the type of the hydrocarbon substituent and its number of carbon atoms, the siloxane chain length or the ratio of dimethylsiloxane to C2-C60-alkylsiloxane units there is a special need for polymers having high siloxane chain length and long chain alkyl siloxy substituents in one molecule.

The U.S. Pat. No. 6,211,323 describes two processes for the preparation of a high molecular weight specific poly(alkylmethyl-alkylaryl)siloxane terpolymers having a low SiH content. This disclosure relates to the manufacturing of triorganosiloxy-endblocked poly(methyl(C6-C40-alkyl)-siloxane)-poly(methyl(aralkyl)siloxane)-poly(methyl(C2-C4-alkyl)-siloxane)-terpolymers.

U.S. Pat. No. 5,516,870 discloses a method of making higher alkyl methyl cyclic siloxanes comprising the steps (i) forming a reaction mixture containing an alpha-olefin, one or more silanol-free methylhydrogen cyclic siloxanes, and less than about 100 parts per million water, (ii) contacting the essentially anhydrous silanol-free reaction mixture with anhydrous platinum supported on carbon catalyst, (iii) agitating the mixture and catalyst to form an alkylmethyl cyclic siloxane, and (iv) continuing the reaction until the alkylmethyl cyclic siloxane is SiH free; where “SiH free” intended to mean that the amount of hydrogen present as SiH is below the detection limit of Fourier Transform Infrared Spectroscopy.

U.S. Pat. No. 5,554,708 and U.S. Pat. No. 5,578,692 disclose a method of making linear triorganosiloxy endcapped methylhydrogen polysiloxanes having chain length of up to 2156 comprising the steps (i) forming a reaction mixture containing a silanol-free hexaorganodisiloxane, one or more silanol-free methylhydrogen cyclic siloxanes, and less than about 100 parts per million water, (ii) contacting the reaction mixture with anhydrous trifluoromethane sulfonic acid catalyst, and (iii) agitating the mixture and the catalyst at below 100° C. to form a linear triorganosiloxy endcapped methylhydrogen polysiloxane. This product is used as precursor for the hydrosilylation with a C10-olefin.

U.S. Pat. No. 4,652,386 A claims structures of a lubricating oil component miscible in mineral oils of the formula:

R₃SiO-[MeRSiO]ₐ-[MeR′SiO]_{b}—SiR₃

wherein R═C6-C18 alkyl,
R″=methyl or phenyl,
a=1-225,
b=9−450 (wherein b/a can be 0, 04:1 to 2:1-450:1).

The patent discloses some short chain siloxanes obtained by adding olefins to methyl-hydrogen-dimethylsiloxane copolymers via hydrosilylation. The polymerization reaction was carried out using an acid catalyst. The rheological and lubricating properties of the C6-C18 copolymers have been reported.

Conventionally prepared long chain (C6-C60)-alkylmethyldimethylsiloxane copolymers having more than 450 siloxy units suffer from the problem that they upon storage underlie condensation reaction resulting in an undesired viscosity increase. This happens also in aqueous emulsions comprising the same, which undesirably leads to phase separation and instability of the aqueous emulsions, which are mainly used in the manufacture of cosmetics, polishing agents, antifoam compositions etc.

Accordingly, there has been a strong desire to reduce the tendency of condensation or cross-linking of the long chain (C6-C60)-alkylmethyl-dimethyl-siloxane copolymers, in particular, in order to increase their storage stability and also the storage stability of the aqueous emulsions comprising them. The prior art processes of preparing such poly(C6-C60)-alkylmethylsiloxanes having higher molecular weights and long alkyl chain length involve the manufacture of high molecular weight Si—H-group containing polyorganosiloxanes generated by acidic equilibration reactions of lower molecular weight polyhydrogenorganosiloxanes and subsequent hydrosilylation with higher alpha olefins or other unsaturated hydrocarbons. This process suffers however from a number of disadvantages due to the hydrosilylation in the last stage of the process. First of all the hydrosilylation catalyst cannot be removed easily from the end product having a high viscosity. The presence of the hydrosilylation catalyst and possibly of the olefins or side products formed therefrom in the end product may cause problems such as discoloration, reduced transparency etc., which may lead to use restrictions, especially in cosmetic applications. Further it is difficult in a process wherein a high molecular polyhydrogenmethylsiloxane is used as precursor to achieve a high conversion of the SiH groups so that in the final product only very small traces of SiH groups remain. Furthermore, is has been observed that poly(C6-C60)-alkylmethyl-dimethylsiloxanes prepared by such conventional route suffer from stability problems, possibly due to impurities containing in the end product. The attempt to overcome these problems by using highly pure starting materials is costly, and the problem of the discoloration due to residual catalysts remains. Accordingly is has been a strong desire of the present inventors to overcome such prior art problems and to improve the properties of (C6-C60)-alkylmethyldimethylsiloxanes to thereby extend their scope of applicability. Furthermore, it is an object of the present invention to provide a method of making trialkyl siloxy endcapped, long chain (C6-C60)-alkyl-methyldimethylsiloxane copolymers having improved stability i.e. constant viscosity over a long time, in particular, in the presence of heat, water and/or emulsifiers especially polyether derivatives having hydroxyl groups.

As a result of extensive efforts of the present inventors, there have been found new routes to poly(higher)alkylmethylsiloxanes which surprisingly lead to improvements in the properties of such poly(higher)alkylmethylsiloxanes and also to simplifications in the process of manufacturing them.

### SUMMARY OF THE INVENTION

Surprisingly the inventors have foand, that with processes for making polyorganosiloxanes comprising (C6-C60)-alkylmethylsiloxy-groups and dimethylsiloxy groups, comprising the steps
a) hydrosilylation of an C6-C60-olefin with a SiH-group-containing-polyorganosiloxane in the presence of a hydrosilylation catalyst, comprising optionally a filtration step,
b) subjecting the reaction product obtained in step a) to the reaction with at least one polydimethylsiloxane in the presence of a basic catalyst or phosphoronitrile chloride,
c) optionally neutralizing the catalyst used in step b),
d) optionally separating low volatiles from the reaction product obtained, (in the following referred to as the first embodiment of the invention)
or
e) hydrosilylation of an C6-C22-olefin with a SiH-group-containing-silane in the presence of a hydrosilylation catalyst,
f) subjecting the reaction product of step e) to polyorganosiloxane-formation, comprising optionally a filtration step,
g) subjecting the reaction product obtained in step f) to the reaction with at least one polydimethylsiloxane in the presence of a basic catalyst or phosphonitrilic chloride,
h) optionally neutralizing the catalyst used in step g),
i) optionally separating low volatiles from the reaction product obtained, (in the following referred to as the second embodiment of the invention)
   long chain (C6-C60)-alkylmethyldimethylsiloxanes with improved stability can be obtained.

### DETAILED DESCRIPTION OF THE INVENTION

The (C6-C60)-alkyl group in the term “polyorganosiloxanes comprising (C6-C60)-alkylmethylsiloxy-groups and dimethylsiloxy groups” may include linear, branched or cyclic C6-C60-alkyl, preferably linear C6 to C20-alkyl or C8 to C12-cycloalkyl. (C6-C60)-alkyl may also include substituted (C6-C60)-alkyl groups.

In step a) of the first embodiment of the invention the hydrosilylation of an C6-C60-olefin with a SiH-group-containing-polyorganosiloxane in the presence of a hydrosilylation catalyst is carried out.

The SiH-group-containing-polyorganosiloxane is preferably selected from trialkyl-siloxy-endcapped siloxanes comprising methylhydrogensiloxy and/or dimethyl-siloxy groups, or cyclic siloxanes comprising methylhydrogensiloxy and/or dimethylsiloxy groups. These SiH-group-containing-polyorganosiloxanes are usually prepared via the known acid catalyzed polymerisation of hydrolysates of methylhydrogenchloro-/alkoxysilanes and optionally trialkylsiloxy- and/or dialkylhydrogensiloxy-endcapped siloxanes and/or dimethylsiloxanes and/or cyclic methyl-hydrogensiloxanes and/or cyclic dimethyl siloxanes.

For example, such SiH-group-containing-polyorganosiloxanes have the general formula (I):

(Me)ᵣHₛSiO-[MeHSiO]ₙ-[Me₂SiO]ₘ—Si(Me)ᵣHₛ (Ia)

wherein
Me is CH₃ (this applies for the entire specification),
r is 2 or 3,
s is 0 or 1,
n is 1 to 300, preferably 1 to 200, more preferably 1 to 100
m is 0 to 400, preferably 1 to 200, more preferably 0 to 100.

The siloxy units may have a random or blockwise distribution.

More preferably the SiH-group-containing-polyorganosiloxanes have the general formula:

(CH₃)ᵣHₛSi—O—[(CH₃)HSiO]₁₋₁₀₀Si(CH₃)ᵣHₛ (Ib)

r, s and n are as defined before.

Also applicable are cyclic methylhydrogen siloxanes of the formula (Ic)

[MeHSiO]ₚ (Ic)

with
p=3 to 5, preferably 4.

Such cyclic methylhydrogen siloxanes are in particular prepared by depolymerisation of hydrolysates of methylhydrogendichlorosilanes. The cyclic methylhydrogen silo-xanes can be purified by distillation.

Also mixtures of linear and cyclic methylhydrogen siloxanes are applicable in step a) of the first embodiment of the invention. And if desired new polymers can be gene-rated by acid catalyzed reaction between (Ia), (Ib) or (Ic).

Due to their process of preparation polymers of the formulas (Ia) or (Ib) can include the residual reaction product of the acid catalyst and a neutralization agent in amounts of 10-10000 ppm per siloxane.

However, it is preferred to choose hydrogen siloxane compounds of the formula (I) which contain a low content of residual reaction product of the catalyst and the neutralization agent.

Therefore especially preferred are compounds (Ic).

Also applicable in this respect are compounds which are selected from the group of (Ia) and (Ib), which are prepared with LPNC (Linear PhosphoroNitrile Chloride) or acid catalysts on carriers, because LNPC can be used in very small amounts and catalysts on carriers can be separated easily. Acid catalysts on carriers are selected from the group of resins having sulfonic acid groups on the surface like, Lewatit®, Dowex®, Nafion®, acid activated clays or carbons which can particularly be used.

The SiH-group-containing-polyorganosiloxane to be used in step a) preferably has a SiH-group content of more than 50 mol-% based on the total amount of silicon atoms. More preferably the SiH-concentration is more than 70 mol. %, especially preferred the SiH-content is more than 85 mol. %. The high concentration of SiH-groups in the precursor results in a high yield in the hydrosilylation reaction.

The SiH-group-containing-polyorganosiloxane preferably has a degree of polymerization of 1 to 300, preferably 1 to 200, most preferably the chain length includes 3 to 100 siloxy units.

The C6-C60-olefins to be used in step a) are suitably selected from linear or branched alpha-olefins, cyclic olefins or mixtures thereof. In particular, the C6-C60-olefin is selected from alpha-olefins selected from the group consisting of 1-hexene, 2-methyl-1-pentene, 3-methyl-1-pentene, 4-methyl-1-pentene, 1-heptene, 2-methyl-1-hexene, 1-octene, 2-methyl-1-heptene, 1-nonene, 1-decene, 1-andecene, 1-dodecene, 1-tri-decene, 1-tetradecene, 1-pentadecene, 1-hexadecene, 1-octadecene, 1-nona-decene, 1-eicosene, hexacosene, octacosene, triacontene, tetratriacontene, hexa-triacontene, octatriacontene, tetracontene, dotetracontene, tetratetracontene, hexa-tetracontenen, octatetracontene, pentacontene, dopentacontene, tetrapentacontene, hexapentacontene, octapentacontene and hexacontene and mixtures thereof, and from cyclic olefins selected from cyclohexene, vinylcyclohexane, vinylcyclohexene, limonene, norbornene, ethylidene norbornene and dicyclopentadiene, and mixtures thereof.

In case that the polyorganosiloxanes comprising (C6-C60)-alkylmethylsiloxy-groups and dimethylsiloxy groups, comprise fluorinated polyorganosiloxanes can be obtained by using fluorinated olefins like perfluoroalkylethylene, and perfluoroalkyl(poly)ether.

The hydrosilylation reaction of step a) (and similar the hydrosilylation step e)) can be started with any of the known hydrosilylation catalysts preferably at temperatures between 20 to 200° C. and 0.001 to 100 bar. The reaction between 50 to 150° C. at 0.1 to 10 bar, more preferably at normal pressure (1030 mbar) is particularly preferred.

Preferred hydrosilylation catalysts are selected from one or more transition metals or transition metal compounds, wherein the transition metal compound are selected from the group, consisting of platinum, rhodium, iridium, palladium, nickel and ruthenium, or mixtures thereof.

The transition metal catalyst can be used as metal or as complex compound and/or as salt thereof. The catalysts can be used with and without carriers, in a colloidal or powdery phase.

It is preferred to use platinum or compounds thereof as hydrosilylation catalyst for the addition of the olefins to the SiH-precursor in step a).

The amount of especially the platinum catalyst is generally in the range of 0.1-1000 ppm, calculated as metal, related to the weight of the SiH-siloxane.

In a preferred embodiment the metal of the catalyst is in the range of 1-50 ppm related to the weight of the SiH-siloxane.

The catalyst of the group consisting of Pt, Rh, Ir, Pd, Ni and Ru compounds, their salts and complexes are preferred. Especially the catalyst is selected from the group which consists of Pt-(II), Pt-(IV) Pt-(O)-complex compounds. Especially preferred are the Pt-(0)-complex compounds of olefins, like vinylsiloxanes, e.g. 1:1-complexes of 1,3-divinyltetramethyldisiloxane and/or tetravinyltetramethyl-tetracyclosiloxane, carbon-monoxide complexes (see U.S. Pat. No. 3,865,858), and complexes comprising ethylene, cyclopentadiene, cyclooctadiene or cyclohexene.

U.S. Pat. No. 3,715,334 or U.S. Pat. No. 3,419,593 are incorporated by reference in respect to the vinylsiloxane catalyst based on Pt-(0)-olefin complexes. Such catalysts can be prepared by the reduction of hexachloroplatinum acid or other platinum chlorides in the presence of an alcohol and vinylsiloxanes.

The reaction product of step a) is generally a product having a viscosity below 1000 mPa·s at 25° C., preferably below 500 mPa·s at 25° C. which advantageously allows the separation of the catalyst and discoloring side products resulting in step a) by a simple filtration step. The process of filtration is usually carried out using a porous filter material, which can separate at least particles greater than 2 μm. Optionally an adsorbing filtration aid can be added like charcoal or diatomaceous earth.

The remaining, non-reacted olefins and optionally low boiling siloxanes may be stripped off by distillation at normal pressure or under vacuum preferably below 200° C.

The intermediates made in step a) (and essentially the same applies for the intermediates obtained in step e)) usually have one of the general formula (IIa) to (IIc):

(IIa):

(Me)ᵣR¹ₛSiO-[MeR¹SiO]ₙ-[Me₂SiO]ₘ—Si(Me)ᵣR¹ₛ (IIa)

wherein
Me is CH₃ (this applies for the entire specification),
R¹ is optionally substituted (C6-C60)-alkyl,
r is 2 or 3,
s is 0 or 1,
n is 1 to 300, preferably 1 to 100
m is 0 to 400, preferably 1 to 100.
preferably (IIb):

(CH₃)ᵣR¹ₛSi—O—[(CH₃)R¹SiO]₁₋₅₀Si(CH₃)ᵣR¹ₛ (IIb)

wherein
r, s and n are as defined before,
R¹ is optionally substituted (C6-C60)-alkyl,

The siloxy units may have a random or blockwise distribution.

and cyclic methylalkylsiloxanes of the formula (IIc):

[MeR¹SiO]ₚ (IIc)

with
p=3 to 5, preferably 4.
wherein R¹ is defined above.

### Step e):

Step e) of the second embodiment of the inventive process comprises also hydro-silylation of a C6-C60-olefin with a SiH-group containing silane in the presence of a hydrosilylation catalyst. Therefore it is dealt with such step in the context of step a). As to the C6-C60-olefins, the hydrosilylation catalysts and the hydrosilylation conditions it can be referred to the description of step a) above, but the hydrosilylation step starts from different precursors.

Suitable SiH-group-containing-silanes to be used in step e) include for example: methyldichlorohydrogensilane, dimethylchlorohydrogensilane, hydrogen(trialkoxy)-silane, methylhydrogendialkoxysilane, and hydrogentrichlorosilane, wherein the alkoxy group is preferably methoxy or ethoxy.

Both hydrosilylation reaction steps a) and e) are preferably carried out such that the remaining SiH-content of the hydrosilylation product obtained is below 100 ppm based on the total reaction product, measured by FT IR-spectroscopy.

The hydrosilylation in step a) or e) can be carried out under the assistance of a low boiling solvent which does not participate to the reaction. Solvents are preferably selected from the group consisting of C4-C10 linear, branched or cyclic aliphatic hydrocarbons, C6-C10 aromatic hydrocarbons, both can be substituted with chlorine, fluorine or can contain oxygen resulting in the formation of ether solvents.

The optional filtration in step a) (or f)) can optionally be carried out with solvents in order to increase the filtration rate.

### Step f):

Step f) comprises subjecting the reaction product of step e) to polyorganosiloxane-formation. Usually step f) is carried out by subjecting the (C6-C60)-alkyl-substituted silanes obtained in step e) to polyorganosiloxane-formation by aqueous hydrolysis, optionally by intermediate alcoholysis. Hydrolysis or alcoholysis is carried out in a manner known to the skilled person in the art.

### Step b) or q):

Step b) or g) each comprise subjecting the reaction product obtained in step a) or f) to the reaction with at least one polydimethylsiloxane in the presence of a basic catalyst or phosphoronitrile chloride. Such process is usually referred to in the art as equilibration or co-equilibration process, which involves thermodynamically controlled re-arrangement reactions of siloxy groups.

The polydimethylsiloxane used in steps (b) or (g) is preferably selected from cyclic, linear, or branched polydimethylsiloxanes.

The steps (b) or (g) include the equilibration of the reaction products obtained in step a) or f), that is, in particular siloxane intermediates of formula (II), and any of the polymethylsiloxanes of formula (III):

R₃SiO(R₂SiO)ᵥSiR₃ (IIIa)
v=1 to 3000,
where R is methyl,

(R₂SiO)_{w} (IIIb)
w=3-6.
where R is methyl.

(Here and in the entire specification indices, like ‘v’ or ‘w’ etc. used in the general polysiloxane formulas refer to the number average values, in particular determined by gel permeation chromatography with polystyrene as standard).

Cyclic polydimethylsiloxanes of formula (IIIb) are preferably selected from the group, which consists of hexamethylcyclotrisiloxane, octamethylcyclotetrasiloxane, decamethylcyclopentasiloxane or mixtures thereof. Octamethylcyclotetrasiloxane is particularly preferred.

Linear polydimethylsiloxanes (IIIa) are preferably selected from the group which consists of trialkylsilyl endcapped polydimethylsiloxanes of the formula (IIIa), wherein the index “v” is 0 to 2500, more preferably v=2 to 100.

Mixtures of linear polydimethylsiloxanes (IIIa) and mixtures of cyclic polydimethyl-siloxanes (IIIb) and mixtures of linear and cyclic polydimethylsiloxanes can be used as well.

Branched polydimethylsiloxanes include cyclic or linear polydimethylsiloxanes having preferably not more than 0.5 mol. % branching units of the type RSiO_{3/2} or SiO_{4/2}.

If the poly(C6-C60-alkyl)methylsiloxane obtained in step a) or f) is linear, like those shown in formula (IIa) or (IIb) the equilibration step b) or g) is preferably carried out with cyclic polydimethylsiloxanes (IIIb).

If the poly(C6-C60-alkyl)methylsiloxane obtained in step a) or f) is cyclic, like those shown in formula (IIc) the equilibration step b) or g) is preferably carried out with linear polydimethylsiloxanes (IIIa).

If the equilibration step b) or g) is carried out with cyclic polydimethylsiloxanes preferably they are used in a mixture with at least one trialkylsilyl-endcapped linear polydimethylsiloxane, in order to provide an appropriate amount of terminating units.

The equilibration steps b) or g) are carried out in the presence of at least one catalyst selected from basic catalyst and phosphoronitrile chloride.

Surprisingly the inventors found that the stability of the polymer as a result of the equilibration reaction is improved if a basic catalyst is employed. If the usual acid equilibration catalyst is used in step b) or g) the stability of the final product is inferior.

In the present invention phosphoronitrile chlorides refer to a compound comprising —N═PCl₂— units. Preferably the phosphoronitrile chlorides have more than two of these units.

Basic catalysts used in steps b) or g) include preferably alkaline metal hydroxides, for example selected from potassium hydroxide, rubidium hydroxide, and cesium hydroxide, alkaline metal siloxanolates, tetraorganoammonium hydroxides, and tetraorganophosphonium hydroxides, wherein in each organohydroxide, the organo group preferably includes C1 to C8-alkyl, phenyl and/or benzyl.

These catalysts are preferably dispersed or solved in a low viscous siloxanes. Preferably a co-solvent is used, selected for example from DMSO (dimethyl sulfoxide), crown ethers, polyethers, hexamethylene phosphoric acid triamide. The co-solvent can act as co-catalyst in order to accelerate the equilibration reaction.

If alkaline metal hydroxides are used they can be reacted with siloxanes to form alkaline metal siloxanolates by appropriate heating and subsequent dehydration. This is advantageous because the siloxanolates are soluble in siloxanes, and because the siloxanolates have a lower water content.

The basic catalysts and the phosphoronitrile chlorides are preferably used in an amount of 2 to 2000 ppm calculated on the basis of total amount of siloxanes involved in the equilibration reaction. The level of necessary catalyst depends also on the amount of remaining acidic residues in the products obtained in step a) or f). Therefore the amount must be adjusted by controlling the free basic catalyst. This can be controlled either by a titration in an aqueous medium like water/alcohol and appropriate acid/bases-indicators or by measuring the increasing polymerization degree by GPC or the viscosity.

The temperature range of the equilibration is preferably 20 to 200° C., preferably over a period of 0.2-5 h.

Particularly preferred temperatures are 20-150° C. for phosphoronitrile chloride catalysts. Particularly preferred temperatures are 20-80° C. for ammonium or phosphonium catalysts. Particularly preferred temperatures are 130-180° C. for alkaline metal catalysts and siloxanolates. Alkaline metal catalysts, i.e. hydroxides or siloxanolates are preferred. Most preferred are potassium and cesium siloxanolates.

Steps (b) or (g) are preferably carried out in a temperature range of 40 to 180° C. and at normal pressure (1030 mbar).

### Step c) and h):

Optionally the catalyst can be deactivated in step c) or h), which comprises neutralisation of phosphoronitrile chloride or the basic catalyst or thermal deactivation of the basic ammonium or phosphonium catalyst. Preferred compounds for the neutralization are mineral acids, like H₂SO₄, H₃PO₄, or partial esters thereof, or partial siloxanolates thereof, reaction products of P₄O₁₀ with siloxanes, sulfonic acids and the siloxanolates thereof, HCl, chlorosilanes or chlorosiloxanes, mono-, di- or tris-chloroethylphosphite and the like.

Preferably the neutralization takes place at temperatures between 20-180° C. suitably over 0.2-2 h. If an alkaline catalyst has to be deactivated a molar ratio of 0.2-1 acidic equivalents measured in an aqueous environment (water/alcohol) per equivalent alkali used in the polymerization. The phosphoronitrilie chloride is neutralized for example with ammonia gas or primary or secondary C1-C10-aliphatic and/or aromatic amines with a mol ratio of approximately 1:1. The basic ammonium or phosphonium catalysts are suitably deactivated by increasing the temperature up to 80-200° C. optionally under assistance of inert gases or vacuum 100-900 mbar.

If alkaline metal catalysts, i.e. hydroxides or siloxanolates, are used as equilibrations catalysts the preferred neutralization agent is selected among mineral acids, chloro-siloxanes and/or tris-(2-chloroethyl)phosphite.

step d) and i):

The optionally steps d) or i) comprise the separation of low boiling molecules. These are in particular residues or side products of the olefins, or siloxanes having low boiling points, i.e. compounds having low molecular weight, i.e. a polymerization degree of less than 6.

The evaporation is carried generally at 80-200° C. at 10-1030 mbar over 0.2-2 h.

As a result of step c) and optionally d) or g) and optionally i) a linear product of the formula (IV) can be obtained:

(CH₃)ᵣR¹ₛSi—O—[(CH₃)(R¹)SiO]_{y}[(CH₃)₂SiO]ₓSi(CH₃)ᵣR¹ₛ (IV)
wherein
r is 2 or 3,
s is 0 or 1,
R¹ is (C6-C60)-alkyl,
x+y>500, and x≧450, y≧1
1000>x/y>1.5.

The siloxy units may have a random or blockwise distribution. Preferred is random distribution.

In particular, for cosmetic formulations compounds of the formula

(CH₃)ᵣR¹ₛSi—O—[(CH₃)(R¹)SiO]_{y}[(CH₃)₂SiO]ₓSi(CH₃)ᵣR¹ₛ (IV′)
wherein
r is 2 or 3,
s is 0 or 1,
R¹ is optionally substituted (C6-C60)-alkyl,
x>500, and
1000>x/y>1.5, preferably 200>x/y>20, more preferably
100>x/y>50
   are preferred, because these compounds have a higher substantivity on surfaces, in particular, in hair care applications.

Particularly preferred are polyorganosiloxanes of formula (IV′) wherein x+y is >600, more preferred, wherein x+y is >1000 and for some applications x+y is >1200.

Particularly preferred are polyorganosiloxanes of formula (IV′) wherein x>800, more preferred wherein x>1000.

These compounds may contain small amounts of branched products, containing T and/or Q-units.

Copolymers of this class with high degree of polymerization Pₙ (chain length x and y) (as well as correspondingly high viscosities) show particular performance properties in some of these applications.

Standard synthetic pathways led to inconsistent results and gelling due to lack of stable polymer structures over the time. Thus the present invention solves the problem of providing high molecular weight, high viscosity copolymers of this class with a high storage stability by the new process.

The compounds of the invention provide specific solubility parameters, melting points, viscosity parameters which enables the design of new properties of emulsions or solutions having superior properties in particular for surface treatment, where high substantivities must be achieved.

The poly(C6-C60)-alkylmethyl-dimethylsiloxanes exhibits modified solubility properties due its C6-C60-alkyl substituents.

The character can be changed towards a higher solubility in fatty acids, alcohols or fatty acid esters by increasing the ratio of the R¹CH₃SiO to the (CH₃)₂SiO— groups, for example to

100>x/y.

These properties can advantageously be used for cosmetic formulations or treatment and care of leather and wooden furnitures or polishing. The higher melting points of some of the compounds are also helpful in order to provide thickening agents or viscosity index modifiers in engine oils or creams and pastes for example for leather and hard surface treatments.

Especially in cosmetic formulations for hair and skin care the poly(C6-C60)-alkyl-methyl-dimethylsiloxanes can broaden the application range and possible uses of the silicone compounds. One preferred application is the as conditioner for hairs. Conditioner means that the poly(C6-C60)-alkylmethyl-dimethylsiloxanes improve the gloss, volume and/or smoothness of the hair. Another application is the use in lipstick compositions having improved gloss and water resistance.

A preferred composition comprising poly(C6-C60)-alkylmethyl-dimethylsiloxanes are aqueous emulsions or mixtures with hydrocarbons. The improved stability of the poly(C6-C60)-alkylmethyl-dimethylsiloxanes enables to produce aqueous emulsions with higher stability and hydrocarbon mixtures without phase separation in particular upon storage.

Accordingly the present invention also provides the use of the polyorganosiloxanes in particular prepared by the process of the present invention in and/or as lubricating compositions, leather care compositions, polishing compositions, foam stabilizers, anti-foam agents, rheological additives, like thickening agents, for example in paint compositions, release agents, in particular, mold release agents, high refractive index additives, and personal care compositions, like cosmetic formulations. Likewise the present invention provides the use of the polyorganosiloxanes as a cosmetic skin or hair conditioner, lubricant, polishing agent, viscosity modulating agent, or as dust preventing additive, for example for pigment powders. The present invention also relates to aqueous emulsions, comprising the polyorganosiloxanes according to the invention and prepared according to the invention, respectively. In further particularly preferred embodiment these aqueous emulsions, comprise in addition at least one emulsifier.

The poly(C6-C60)-alkylmethyl-dimethylsiloxanes compounds preferably used in hair care have a ratio of Me₂SiO to (C6-C60)-(alkyl)MeSiO of x:y of about from 99:1 to about 80:20.

If structures of poly(C6-C60)-alkylmethyl-dimethylsiloxanes having higher alkylsiloxy contents are applied the treated hair could have a greasier appearance. The composition wherein the poly(C6-C60)-alkylmethyl-dimethylsiloxanes are being preferably be used are emulsions comprising emulsifiers and surfactants or one phase compositions comprising hydrocarbons and emulsifiers. General speaking the poly(C6-C60)-alkylmethyl-dimethylsiloxanes must be miscible in a cosmetically acceptable carrier medium. The term “cosmetically acceptable” means that it is suitable for contact with the human body, skin and hair. Examples of such compositions are represented by typical recipes below.

The inventive process for the manufacturing of poly(C6-C60)-alkylmethyl-dimethyl-siloxanes provide siloxane polymers which enables better storage stability also of such compositions.

The poly(C6-C60)-alkylmethyl-dimethylsiloxanes for hair conditioning agent are preferably applied in amount of ranges from 0.03% to 7%, more preferred are 0.5% to 3 by weight of the composition.

The melting point and solubility can be altered in wide range by changing the numbers of C-atoms in alkyl chain i.e. the chain length and the amounts of (C6-C60)-alkyl substituted siloxy units. By this way the character of the poly(C6-C60)-alkylmethyl-dimethylsiloxanes can be modified between relative low viscous polymethylsiloxane having low melting points and poly(C6-C60)-alkylmethyl-dimethylsiloxanes having higher melting points and stronger similarity to paraffins.

The preferred melting behaviour of the poly(C6-C60)-alkylmethyl-dimethylsiloxanes of the present invention enables to achieve polyalkylmethylsiloxanes having the form of a fluid or a gel- or cream-like material rather than a solid or a wax at room temperature. In such cases this character is not the result of crosslinked structures caused by undesired reaction upon storage as known from prior but part of structural properties of the inventive poly(C6-C60)-alkylmethyl-dimethylsiloxanes.

The melting points of the inventive poly(C6-C60)-alkylmethyl-dimethylsiloxanes in detail can have a range of about −40° C. up to 120° C. measured by a differential scanning calorimeter.

Higher melting points are suitably realized by alkyl substituents having more than 10 carbon atoms preferably more than 12 carbon atoms and an x:y of less than 97:3.

Preferred carriers for emulsions are water or C2-C3-alcohols. The emulsifiers preferably used in the manufacturing of emulsions and to stabilize the discontinue or continue phase of poly(C6-C60)-alkylmethyl-dimethylsiloxanes comprise:

Surfactants as ingredients for cosmetic formulations are described in A. Domsch: Die kosmetischen Präparate, Verlag für Chem. Industrie, 4th Edition, 1992, in Kosmetikjahrbuch 1995, Verlag für chemische Industrie, 1995, and H. Stache, Tensidtaschenbuch, 2nd Edition, Carl Hanser Verlag, 1981.

a) Anionic surfactants or detergents and inorganic salt can be examplified without any limitation selected from the group which consists of sodium and fatty and alkylsulfates, alkylethersulfates, alkarylsulfates, olefinsulfonates, ammonium lauryl sulfates acid sodium and ammonium lauryl ether sulfates, alkyl-amidether-sulfates, acylisothiocyanates, acylglutamates, alkylethercarboxylates, alkyl ether carboxylates, methyltaurides and taurides, sarcosides, sulfosuccinates, fatty alkyl ether sulfosuccinates, condensates of proteins and fatty acid, alkylphosphates and alkyletherphosphates.

Wherein the free acids as well as its alkaline salts, such as magnesium-, ammonium- and mono-, di- and triethanolamine salts can be used.

The alkyl- and acyl groups consists typically of substituents having 8-18 carbon atoms and are saturated or unsaturated, linear or branched. The alkylthersulfates, alkylamid-ether sulfates, alkylethercarboxylates and alkyletherphosphates can contain 1-10 ethyleneoxide- or propylenoxide-units or a combination thereof and optionally an amount of an acid and water for adjusting the pH-value.

### Amphoteric Surfactants:

Can be examplified without any limitation selected from the group which consists of alkylbetaines, alkylamidobetaines, sulfobetaines, acetates and diacetates, imidazolines, propionates and alkylaminoxides and optionally an amount of an acid and water for adjusting the pH-value.

The alkyl- and acyl groups contain substituents having 8-19 carbon atoms.

b) Nonionic surfactants can be examplified without any limitation selected from the group which consists of alkylethoxylates, arylethoxylates, ethoxylated esters, castor oil ethoxylates, polyglycol amides, polysorbates, fatty alkyl ethers and sorbitan esters of fatty acids, glycerine fatty acid-ethoxylates, alkylphenol polyglycolethers and sugar surfactant such as e.g. alkylglycosides and optionally an amount of an acid and water for adjusting the pH-value.

c) Cationic surfactants can be examplified without any limitation selected from the group which consists of fatty alkyl amidoamines and fatty alkyl quaternary ammonium, compound and. The term “fatty alkyl” is intended to mean a long chain alkyl radical having from about 12 to 22 carbon atoms.

One type of cationic surfactants are:

Monoalkylquats, dialkylquats, trialkylquats, tetraalkylquats, benzylammonium-salts, salts of pyridinum, alkanolammonium, imidazolinum, oxazolinum, thiazoline derivatives, amine oxides, sulfones, whereas the term “quat” implies the presence of at least one quaternary ammonium group.

Cationic polymers are preferably chosen for “2-in-1”-shampoos whereas the cationic polymers can be used beside the previously mentioned ‘pure’ cationic surfactants.

An extended description of this class of polymers are referred in U.S. Pat. No. 5,977,038 and WO 01-41720. Cationic polymers can be examplified without any limitation selected from the group which consists of cationic protein derivatives, hydroxyl-alkylcellulosis ethers and cationic ‘Guar’-derivatives. Particularly preferred are cationic ‘Guar’ derivatives having the names according to CTFA Guar Hydroxypropyl-trimonium Chloride. These types are commercially available under the brandname Cosmedia Guar C 261 (Henkel), Diagum P 5070 (Diamalt), Jaguar C-Typen and Jaguar EXCEL of Rhodia.

### Auxilliary Additives:

Auxilliary additives for the cosmetic compositions are described in: A. Domsch, Die kosmetischen Präparate, Verlag für Chem. Industrie, 4. edition, 1992; and in: Kosmetikjahrbuch 1995, Verlag für Chemische Industrie, 1995.

Auxilliary additives can be exemplified without any limitation selected from the group which consists of inorganic and organic acids, bases and buffers, salts. Another category are solvents and carriers for active material selected from the group which consists of water, alcohols, hydrocarbons and inorganic or organic solids or powders acceptable in cosmetics or home care products, such as e.g. ethanol, isopropanol, ethylenglycol, polyethylenglycol, propylenglycol, polypropylenglycol, glycol ether and glycerine, thicking agents, stabilizers for emulsions such as e.g. Xanthan Gum, preservatives, biocides, foam stabilizers, anti-foam compositions, Pearlgloss and opacifying agents such as e.g. glycol distearates and titanium dioxide, collagene hydrolysates, keratin hydrolysates, silk hydrolysates, anti-dandruff agents such as e.g. zinc pyrithione, salicylic acid, selene disulfide, sulfur and bitumen derivatives, polymeric emulsifiers, vitamines, dyestuffs, ‘UV filter’, bentonites, parfume oils, fragrances, styling polymers, moisturing additives, extracts of plant and further natural or natural identical raw materials.

The inventive poly(C6-C60)-alkylmethyl-dimethylsiloxanes as active material can be combined with other silicone products, which are selected from the group consisting of cyclic, linear and branched polydimethylsiloxanes having a viscosity of 0.65-200,000,000 mPa·s at 25° C. as well as their mixtures such as e.g. octaorgano-cyclotetrasiloxanes, octamethylcyclotetrasiloxanes, decaorganocyclopentasiloxane, dodecaorganocyclohexasiloxanes. These product are commercially available such as SF 1173, SF 1202, SF 1217, SF 1204 and SF 1258 from Momentive Performance Materials formerly GE Bayer Silicones, Dimethicone such the Baysilone M-oils (M3 to M 2,000,000), SE 30, SF 1214, SF 1236, SF 1276 and CF 1251 from Momentive Performance Materials and Dimethiconole, SiOH-endstopped ‘Gums’ from Momentive Performance Materials and DC 1501 or DC 1503 from Dow Corning.

The use of the poly(C6-C60)-alkylmethyl-dimethylsiloxanes in emulsiones with non-ionic, anionic and cationic emulsifiers such e.g. SM 2169, SM 2785, SM 555, SM 2167 and SM 2112 from Momentive Performance Materials in combination with emulsions of the poly(C6-C60)-alkylmethyl-dimethylsiloxanes is in particular preferred. Another improved for hair care application can be achieved by the additional use of known amino- or ammonium functional polyorganosiloxanes, incorporated by reference of the following publications WO 99/44565, WO 99/44567, WO 99/49836, WO 99/53889, WO 97/12594, U.S. Pat. No. 6,028,031, EP 0811371, WO 98/18443, WO 98/43599, WO 02/10257, WO 02/10259 and US 2002-0182161.

Another type of silicones suitable as cosmetic ingredients or carier can be selected from group which consists of solid silicones such as e.g. MQ-resins like SR 1000 of Momentive Performance Materials and its solutions in siloxanes or aliphatic or aromatic solvents, e.g. isododecane.

Another type of silicones suitable as cosmetic ingredients or carrier can be selected from organofunctional silicones, such as alkyl-, aryl-, arylalkyl-, phenyl-, fluoralkyl-, and polyether-modified silicones such as types like SF 1632, SF 1642, SF 1555, Baysilone F 1301, Baysilone PK 20, FF 157, SF 1188A, SF 1288 and SF 1388 of Momentive Performance Materials.

### Ingredients for Hair Dying Compositions:

Dyestuffs and other ingredients of hairdying compositions are described in: A. Domsch, Die kosmetischen Präparate, Verlag für Chem. Industrie, 4. edition, 1992. Dyestuffs are described in: Verordnung über Kosmetische Mittel (Kosmetik Verordnung), Bandesgesetzblatt 1997, Teil I S. 2412, §3 and Anlage 3 and in European Community (EC) Directive, 76/68/EEC, Annex IV.

Typical hair dyeing compounds of dyeing compositions are selected from the group which consists of:

### Permanent Hair Dyeing Compositions:

Permanent hair dyeing compositions which can withstand several cycles of washes (more than 10) and accrue by chemical reaction between dyestuff-precursors under oxidative condition by e.g. the presence of hydrogenperoxide. Within the precursors for dyestuffs it has to be distinguished between oxidizing bases (developer) and coupling components (modifier).

### Oxidizing Bases:

Typical oxidizing bases can be examplified without any limitation of the invention are selected from the group which consists of following components m- and p-phenylendiamine (diaminobenzene), its N-substituted derivates and salts, N-substituted derivates of the o-phenylendiamine, o-, m- and p-toluoylendiamine (methyl-diaminobenzene), its N-substituted derivates and salts, p-amino-diphenylamine, -hydrochlorides and -sulfate, o-, m- and p-aminophenol and -hydrochloride, 2,4-diaminoisosulfate (4-methoxy-m-phenylendiaminsulfate), o-chloro-p-phenylen-diaminsulfate, picramine acid (2,4-dinitro-6-aminophenol) and 2,4-dinitro-1-naphtol-sulfonic acid as well its sodium salts.

### Coupling Components:

Typical coupling components can be examplified without any limitation of the invention are selected from the group which consists of following components hydrochinone (1,4-dihydroxybenzene), resorcine (1,3-dihydroxybenzene), brenzcatechine (1,2-dihydroxybenzene), α-naphtol (1-hydroxynaphtaline), pyrogallol (1,2,3-trihy-droxybenzene) and 2,6-diaminopyridine.

Usually oxidizing bases and coupling bases are mixed together under the assistance of surfactants in ‘oil-in-water’ emulsions, however also simple solution or shampoos are well known as compositions. These composition comprises in addition antioxidants such as e.g. sodium sulfite, sodium dithionite, ascorbic acid or thioglycol acid for stabilizing the presursors and are adjusted with alkaline substances such as e.g. ammonia up to a pH-values of 8 to 12 (preferred 9-11). Further surfactants are added serving as wetting agents, complexing agents for heavy metals, fragrances for superposing the ammonia odour, and solvents such as ethanol, ethylenglycol, glycerine or benzylalcohol.

Typically the permanent hair dyeing compositions are prepared as 2-component systems consisting of dyeing solutions, -creams or -shampoos as described above and of a developer solution. The developer solution herein contains preferably between 6-12 wt. % hydrogenperoxide and can optionally comprise also components of the dying component containing formulation. The peroxide solution has to stabilzed duly.

### Semipermanent Hair Dying Compositions:

Semipermanent hair dying compositions has been developed in order to enable a coloring of hairs which withstand 6-10 washing cycles with shampoo. In this case direct coupling dyestuffs are applied, which essentially selected from the group which consists of nitro-, azo- and anthrachinone dyestuffs. Typically applied formulations are solutions, creams, shampoos or aerosol foams (mousses).

### Temporary Hair Dying Compositions:

Temporary hair dying compositions comprise different to the semi-permanent hair dyeing compositions bigger dyestuff molecules, which are not able to penetrate into the hair. They have been developed to achieve coloring for 1-6 washing cycles. Typically the azo- and basic dyestuffs like azin- and thiazine derivatives are also used herein. Dyestuffs and other ingredients of hair dyeing compositions are described in: A. Domsch, Die kosmetischen Präparate, Verlag für Chem. Industrie, 4th edition, 1992. Dyestuffs are described in: Verordnung über Kosmetische Mittel (Kosmetik Verordnung), Bandesgesetzblatt 1997, Teil I S. 2412, §3 and attachment 3 and in European Community (EC) Directive, 76/68/EEC, Annex IV.

In the preferred field of application of the poly(C6-C60)-alkylmethyl-dimethylsiloxanes are the use in hair care, whereas hair shampoos, hair conditioners such as cream rinses and leave-on products, ‘mousse’-products and hairsprays are especially preferred areas of application.

The shampoos comprising the polymethylalkylsiloxane can be composed as sole hair conditioning agent present in the composition or can be present with other conventional hair conditioning agents such as organic cationic hair conditioning agents which can be added to the shampoos. Preferred hair care compositions include cationic conditioning agents as well as a carrier medium which can be used as a ‘Leave-on’-treatment or it can be used for ‘Rinse-off’ of the hair. Cationic conditioning compounds are selected from the group which consist of stearyl dimethyl benzyl ammonium chloride, cetyl dimethyl amine oxide, cationic polymers selected from the group of cationic cellulosis polymers, cationic arylates, cationic silicone like polyammonium-polyorganosiloxanes like SILFOFT®, SILWET®, Magnasoft®. Other brandnames are Celquat®, Merquat®, Jaguar®, Luviquat® or Gafquat®,

These type are e.g. described in patent application EP-A-0,337,354 and in French patent applications FR-A-2,270,846, 2,383,660, 2,598,611, 2,470,596 and 2,519,863. Other polymers of the quaternary polyammonium, polyaminoamide and polyamine type which can be used are mentioned in FR 2,505,348 or 2,542,997. Among these polymer there are roughly 11 categories:
(1) Quaternized or non-quaternized vinylpyrrolidone/dialkylaminoalkyl acrylate or methacrylate copolymers, such as the products sold under the name “Gafquat®” by the company ISP such as, for example, Gafquat® 734, 755 or HS100 or alter-natively the product known as “Copolymer 937”. These polymers are described in detail in FR 2,077,143 and 2,393, 573.
(2) The cellulose ether derivatives containing quaternary ammonium groups, described in FR 1,492,597, and in particular polymers sold under the names “JR” (JR 400, JR 125 and JR 30M) or “LR” (LR 400, or LR 30M) by the company Union Carbide Corporation. These polymers are also defined in the CTFA dictionary as quaternary ammoniums of hydroxyethylcellulose which has reacted with an epoxide substituted with a trimethylammonium group.
(3) Cationic cellulose derivatives such as cellulose copolymers or cellulose derivatives grafted with a water-soluble monomer of quaternary ammonium, and described in particular in U.S. Pat. No. 4,131,576. The commercial products corresponding to this definition are products sold under the names “Celquat® L 200” and “Celquat® H 100” by the company National Starch.
(4) The cationic polysaccharides described more particularly in U.S. Pat. Nos. 3,589,578 and 4,031,307, such as guar gums containing cationic trialkylammonium groups. Guar gums modified with a salt (e.g. chloride) of 2,3-epoxypropyltrimethylammonium are used, for example. Such products are sold in particular under the trade names Jaguar® C13S, Jaguar C 15, Jaguar C 17 or Jaguar C162 by the company Meyhall.
(5) Polymers consisting of piperazinyl units and of divalent alkylene or hydroxyl-alkylene radicals containing straight or branched chains, as well as the oxidation and/or quaternization products of these polymers. Such polymers are described, in particular, in FR 2,162,025 and 2,280,361.
(6) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound.
(7) The polyamino amide derivatives resulting from the condensation of polyalkylene polyamines with polycarboxylic acids followed by alkylation with difunctional agents. Such polymers are described in particular in FR 1,583,363. Among these derivatives more particularly the adipic acid/dimethylamino-hydroxypropyl/diethylenetriamine polymers sold under the name “Cartaretine F, F4 or F8” by the company Sandoz can be mentioned.
(8) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. Such polymers are described in particular in U.S. Pat. No. 3,227, 615 and 2,961,347. Polymers of this type are sold in particular under the name “Hercosett 57” by the company Hercules Inc. or alternatively under the name “PD 170” or “Delsette 101”.
(9) Cyclopolymers of methyldiallylamine or of dimethyldiallylammonium, such as the homopolymers or copolymers. These polymers are described in particular in FR 2,080,759 or 2,190,406.
   The polymers of the dimethyldiallyl-ammonium chloride homopolymers sold under the name Merquat® 100.
(10) The quaternary diammonium polymer. These polymers generally have a number-average molecular mass of between 1000 and 100,000 g/mol. Polymers of this type are described in particular in FR 2,320,330; 2,270,846; 2,316,271; 2,336,434 and 2,413,907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945 and 4,027,020.
(11) Quaternary polyammonium as described in particular in EP-A-122,324. These products are sold, for example, as Mirapol® A 15, Mirapol® AD1, Mirapol®AZ1 and Mirapol® 175 by the company Miranol.
(12) Homopolymers or copolymers derived from acrylic or methacrylic acids. The comonomer(s) which can be used in the preparation of the corresponding copolymers belong to the family of acrylamides, methacrylamides, diacetone acrylamides, acrylamides and methacrylamides substituted on the nitrogen with lower alkyls, alkyl esters, acrylic or methacrylic acids, vinylpyrrolidone or vinyl esters.
(13) Quaternary polymers of vinylpyrrolidone and of vinylimidazole, such as, for example, the products sold under the names Luviquat® FC 905, FC 550 and FC 370 by the company BASF.
(14) Polyamines such as Polyquart® H sold by Henkel under the reference name “Polyethylene glycol (15) tallow polyamine” in the CTFA dictionary.
(15) Crosslinked methacryloyloxyethyl-trimethylammonium chloride polymers such as the polymers obtained by homopolymerization of dimethylaminoethyl methacrylate quaternized with methyl chloride. This dispersion is sold under the name “Salcare® SC 92” by the company Allied Colloids or sold under the name “Salcare SC 95” by the company Allied Colloids.
   Other cationic polymers which can be used in the context of the invention are polyalkyleneimines, in particular polyethyleneimines, polymers containing vinylpyridine or vinylpyridinium units, condensates of polyamines and of epichlorohydrin, quaternary polyureylenes and chitin derivatives.

Among the cationic polymers which are useful in the context of the present invention, it is preferred to choose quaternary cellulose ether derivatives such as the products sold under the name JR 400 by Union Carbide Corporation, Merquat 100, Merquat 550 and Merquat S by Merck, cationic polysaccharides and more particularly the guar gum modified with 2,3-epoxypropyltrimethylammonium chloride sold as Jaguar C13S by Meyhall.

One purpose of the use of the quaternary ammonium groups present in the organic cationic hair conditioning agents, is to make the poly(C6-C60)-alkylmethyl-dimethylsiloxanes more substantive to the hair than the essentially non-polar polymethylalkylsiloxanes of the present invention. As consequence of matter they can be more efficiently applied upon substrates like hairs or hard surfaces.

Kosmetik Compositions include e.g.:

So-called “Rinse-off” products such as “2-in-1” shampoos, “Body Wash” and hair rinse formulations for the treatment of hairs under the wash or after the dyeing cycle of hairs or the treatment of hairs before the bleaching, the forming of the tresses or derippling, as well as in so-called “Leave-in” products such as hair treatment cures, care creams, hair gels, hair styling products, hair fixatives, and hairs sprays. In addition the formulations include also hair dying compositions.

The hair dying compositions can be distinguished into 3 classes depending on their washing resistance of color persistence: permanent, semipermanent and temporary hair dyeing compositions. The term ‘hairs’ comprises all keratin containing fibers, in particular the human hair. The hair dyeing compositions contain beside the inventive polyalkylmethylsiloxane compounds other usual silicones, cationic polymers, surfactants, auxiliaries and dying compounds. Each of these ingredient can either act ‘per se’ or in combination with other ingredients useful and can serve to improve the volume, the combing properties and the gloss as well as the color permanence after the wash of colored keratinic substrates such as e.g. human and animal hair.

In one embodiments of the preferred compositions the polymethylalkylsiloxanes of the present invention are added to a cream rinse conditioner, which comprises an aqueous emulsion of cetyl alcohol and a fatty alkyl quaternary ammonium compound. In other embodiments of preferred use of the inventive polymers, the poly(C6-C60)-alkylmethyl-dimethylsiloxanes is used together with pressurized gases as a carrier medium which can be dispensed from an aerosol container in form of a foam product so-called “mousse” product or film after the carrier gas is evaporated. The carrier gases can be selected from group which consists of propellant such as propane, butane, N₂ and CO₂.

For all these compositions comprising the polyalkylmethylsiloxane the compositions remain stable and do not separate into several phases which would cause them to be more difficult to apply when dispensed to hair, skin, leather or hard surfaces. The inventive polymethylalkylsiloxane exhibits improved compatibility with a specific compositions and does not suffer from subsequent change of the molecular weight during storage due to post crosslinking or condensation reactions.

### Typical Compositions

The preferred mixtures, emulsions or solutions of the inventive poly(C6-C60)-alkylmethyl-dimethylsiloxanes exhibit the following compositions in wt. % related to the total amount of the composition:

Solutions Resp. Mixtures:

| | |
|---|---|
| 0.1-99.9 wt. % | inventive polyorganosiloxane compounds |
| 0.1-99.9 wt. % | solvents and/or oils and/or silicones, and/or water |

For the manufacturing of the emulsions one can generally use water and nonionic, cationic and amphoteric surfactants or mixtures of surfactants. In addition the composition can comprise auxiliary additives such as inorganic and organic acids, bases and buffer, salts, thickening agents, stabilizers for emulsions like e.g. ‘Xanthan Gum’, preservatives, foamstabilizers, anti-foams and solvents like e.g. alcohols such as ethanol, isopropanol, ethylenglycols, polyethylenglycols, propylenglycols, polypro-pyleneglycols, glycolethers and glycerine and the mixtures thereof.

A preferred emulsion, preferably used for the manufacture of cosmetic formulations consists of the following ingredients in wt.-%, related to the total of the composition:

| | |
|---|---|
| 10-50% | inventive polyalkylmethylsiloxane compound, |
| 1-35% | surfactants, |
| 0-10% | auxilliary agents, |
| 0-20% | solvents, |
| Suppl. to 100% | by water. |

Preferred are microemulsions for cosmetic formulations, the treatment for textiles and other fiber like substrates or the coating of hard surfaces:

Especially preferred is the manufacturing of microemulsions having a high content of active poly(C6-C60)-alkylmethyl-dimethylsiloxanes according to the invention. The concentration of the poly(C6-C60)-alkylmethyl-dimethylsiloxanes content is 5 to 60 wt.-%, preferred are 10 to 50 wt. % related to the total amount of the composition.

A particular preferred microemulsion consists of the following however not limiting components in wt. % related to the total amount of the microemulsion:

| | |
|---|---|
| 20-80% | inventive polyorganosiloxane compound, |
| 0-35% | surfactants, |
| 0-10% | auxilliary agents, |
| 0-20% | solvents, |
| Suppl. to 100% | by water |

A typical inventive shampoo composition for care and conditioning of hairs can be examplified without any limitation of the invention is selected from the group which consists of following components in wt. %, related to the total amount of the composition:

| | |
|---|---|
| 0.01-10% | cationic polymer compound, |
| 2-15% | anionic surfactant, |
| 0-10% | amphoteric surfactant, |
| 0-15% | nonionic surfactant, |
| 0-10% | cationic surfactant, |
| 0-10% | inventive conditioning siloxane (co-adjuvant) |
| 0-10% | auxiliaries |
| up to 100% | suppl. by water. |

A specific shampoo composition for hair can be examplified without any limitation of the invention is selected from the group which consists of following components in wt. %, related to the total amount of the composition:

| | |
|---|---|
| 0.1-12% | cationic surfactant or polymeric compound, |
| 1-35% | sodium or ammonium lauryl-respectively |
| | laurethsulfate (20-30%), |
| 1-6% | cocoamidopropylbetaine (25-35%), |
| 0-3% | guar hydroxypropyltrimonium chloride |
| 0-5% | Polyquaternium-10, |
| 0-12% | inventive silicone conditioning polymer (Co-Adjuvant), |
| 0.01-1% | disodium EDTA, |
| 0.01-1% | phenoxyethynol (and) methylparaben (and) |
| | butylparaben (and) ethylparaben (and) |
| | propylparaben, |
| 0-1% | parfume oil (fragrance), |
| 0-1% | dyestuff, |
| 0-1% | citric acid, |
| 0-2% | sodium chloride, |
| up to 100% | suppl. by water. |

A specific shampoo composition for hair rinse-off can be examplified without any limitation of the invention is selected from the group which consists of following components in wt. %, related to the total amount of the composition:

| | |
|---|---|
| 0.1-15% | cationic surfactant or polymeric compound |
| 0-10% | amphoteric surfactant |
| 0.1-15% | non-ionic surfactant |
| 0-10% | cationic surfactant |
| 0-15% | inventive silicone conditioning polymer (co-adjuvant) |
| 0-20% | auxiliaries |
| up to 100% | suppl. by water. |

Another specific shampoo composition for hair rinse-off can be examplified without any limitation of the invention is selected from the group which consists of following components in wt. %, related to the total amount of the composition:

| | |
|---|---|
| 0.5-15% | cationic surfactant or polymeric compound and (43.5% act. |
| | material in emulsion in water with non-ionic emulsifiers), |
| 0-15% | inventive silicone conditioning polymer (co-adjuvant), |
| 0-10% | cetrimonium chloride (25-35%), |
| 0-3% | guar hydroxypropyltrimonium chloride, |
| 1-10% | cetearyl alcohol, |
| 0-10% | glycerine, |
| 0.01-1% | phenoxyethynol (and) methylparaben (and) |
| | butylparaben (and) ethylparaben (and) |
| | propylparaben, |
| 0-1% | parfume oil (fragrance), |
| 0-1% | dyestuff, |
| 0-1% | citric acid, |
| up to 100% | suppl. by water. |

Another specific shampoo composition for hair care treatment can be examplified without any limitation of the invention is selected from the group which consists of following components in wt. %, related to the total amount of the composition:

| | |
|---|---|
| 0.4-20% | cationic surfactant or polymeric compound, |
| 0-15% | non-ionic surfactant, |
| 0-10% | cationic surfactant, |
| 0-20% | inventive silicone conditioning polymer (co-adjuvant), |
| 0-20% | auxiliaries, |
| up to 100% | suppl. by water. |

Another specific shampoo composition for hair care treatment can be examplified without any limitation of the invention is selected from the group which consists of following components in wt. %, related to the total amount of the composition:

| | |
|---|---|
| 1-20% | cationic surfactant or polymeric compound and (43.5% |
| | act. material in emulsion in water with non-ionic. |
| | emulsifiers), |
| 0.5-10% | stearyl alcohol (and) Steareth-7 (and) steareth-10, |
| 0-20% | inventive silicone conditioning polymer (co-adjuvant), |
| 0-10% | cetrimonium chloride (25-35%), |
| 0-3% | guar hydroxypropyltrimonium chloride, |
| 0-5% | Dimethicone, |
| 0-5% | paraffin oil, |
| 1-10% | stearyl alcohol, |
| 0-10% | glycerine, |
| 0.01-1% | phenoxyethynol (and) methylparaben (and) butylparaben |
| | (and) ethylparaben (and) propylparaben, |
| 0-1% | parfume oil (fragrance), |
| 0-1% | dyestuff, |
| 0-1% | citric acid, |
| 0-2% | sodium chloride, |
| up to 100% | suppl. by water. |

Another specific shampoo composition for hair care treatment can be examplified without any limitation of the invention is selected from the group which consists of following components in wt. %, related to the total amount of the composition:

| | |
|---|---|
| 2-5% | cationic surfactant or polymeric compound (43.5% act. |
| | material in emulsion in water with non-ionic emulsifiers), |
| 0-5% | inventive silicone conditioning polymer (co-adjuvant), |
| 0-2% | Cetrimonium Chloride (25-35%), |
| 0.5-5% | glycerine, |
| 0.25-2.5% | propylenglycol, |
| 0.05-0.2% | parfume oil, |
| 0.1-0.5% | Polysorbat 20, |
| up to 100% | suppl. by water. |

A typical composition for dyeing hair comprising dyestuffs for the temporary, semi-permanent or permanent hair dyeing, care and conditioning of the hairs can be examplified without any limitation of the invention is selected from the group which consists of following components in wt. %, related to the total amount of the composition:

| | |
|---|---|
| 0.1-10% | cationic surfactant or polymeric compound, |
| 1-10% | hair dyestuff precursors or dyestuff depending on the |
| | desired hair color, |
| 0-15% | anionic surfactant, |
| 0-10% | amphoteric surfactant, |
| 0-10% | non-ionic surfactant, |
| 0-10% | cationic surfactant, |
| 0-1% | sodium sulfite, |
| 0-5% | buffer, |
| 0-10% | inventive silicone conditioning polymer (co-adjuvant), |
| 0-10% | auxiliaries, |
| up to 100% | suppl. by water. |

A specific composition for dyeing hair cream can be examplified without any limitation of the invention is selected from the group which consists of following components in wt. %, related to the total amount of the composition:

| | |
|---|---|
| 0.1-10% | cationic surfactant or polymeric compound, |
| 1-5% | hair dyeing precursors or dyestuffs depending on |
| | the desired color of the hair, |
| 2-15% | anionic surfactant, |
| 0-10% | amphoteric surfactant, |
| 0-10% | non-ionic surfactant, |
| 0-10% | cationic surfactant, |
| 0.1-1% | sodium sulfite, |
| 0.1-5% | buffer for pH = 8-12 |
| 0-10% | inventive silicone conditioning polymer (co-adjuvant), |
| 0-10% | auxiliaries, |
| up to 100% | suppl. by water. |

A specific dying solution for permanently dyeing the hair can be examplified without any limitation of the invention is selected from the group which consists of following components in wt. %, related to the total amount of the composition:

| | |
|---|---|
| 0.1-10% | cationic surfactant or polymeric compound and (20% act. |
| | material in emulsion in water with non-ionic emulsifiers), |
| 1-5% | hair dyeing precursors or dyestuffs depending on the |
| | desired color of the hair, |
| 0.1-1% | sodium sulfite, |
| 5-15% | propylenglycol, |
| 5-15% | ammonia (28%), |
| 10-30% | oil aid, |
| 5-15% | isopropanol, |
| 10-30% | alkanol amide, |
| 0-10% | inventive silicone conditioning polymer (co-adjuvant), |
| up to 100% | suppl. by water. |

Another typical dying composition for permanently dyeing the hair can be examplified without any limitation of the invention is selected from the group which consists of following components in wt. %, related to the total amount of the composition:

| | |
|---|---|
| 0.1-10% | cationic surfactant or polymeric compound, |
| 10-30% | hydrogen peroxide (30%), |
| 0-15% | anionic surfactant, |
| 0-10% | amphoteric surfactant, |
| 0-10% | non-ionic surfactant, |
| 0-10% | cationic surfactant, |
| 0-5% | buffer resp. acid for adjusting pH 2-6, |
| 0-10% | inventive silicone conditioning polymer (co-adjuvant), |
| 0-10% | auxiliaries, |
| up to 100% | suppl. by water |

Another typical dyeing composition for permanently dying the hair can be examplified without any limitation of the invention is selected from the group which consists of following components in wt. %, related to the total amount of the composition:

| | |
|---|---|
| 0.1-5% | cationic surfactant or polymeric compound and (20% act. |
| | material in emulsion in water with non-ionic emulsifiers), |
| 10-30% | hydrogen peroxide (30%), |
| 0-5% | inventive silicone conditioning polymer (co-adjuvant), |
| 1-10% | cetearyl alcohol, |
| 0.5-5% | Trideceth-2 Carboxamid MEA, |
| 0.5-5% | Ceteareth-30, |
| 0.5-5% | glycerine, |
| 0.05-2% | pentasodium pentetate (pentasodium diethylentriamine- |
| | penta-acetate, |
| 0.05-2% | sodium stannate, |
| 0.05-2% | tetrasodium pyrophosphate, |
| up to 100% | suppl. by water. |

The inventors have recognized that the inventive compositions or mixtures can be used preferably for manufacturing of cosmetical formulations, such as the treatment, conditioning, cleaning and/or care of colored or substrates to be dyed.

Comparable compositions in particular emulsions can be used as means for, leather care compositions, polishing compositions for hard surfaces like for ceramic, glass, automobiles, anti-foam agents in industrial applications, for example in paint compositions, release agents for adherent and sticky goods like labels or plastic or rubber goods.

The pure poly(C6-C60)-alkylmethyl-dimethylsiloxanes can advantageously be used as lubricating additive in engine oils or on other hard surfaces, as foam stabilizers in polyurethane compositions, rheological additives for thickening or levelling in paint compositions, as anti-dust agent for pigments or mold release agents in the demolding of rubber and plastic goods like tire demolding.

Finally as additive to alter or adjust the refractive index of regular polyorganosiloxanes to higher refractive indices in coating or cosmetic applications.

### EXAMPLES

### Comparison Example 1

A mixture consisting of 1710 g OH-terminated polydimethylsiloxane (PDMS) with a viscosity of 2000 mPa·s at 25° C. and 48 g (0.785 mol SiH)SiH-siloxane having the general structure Me₃SiO(MeHSiO)₅₀SiMe₃ (21 mPa·s at 25° C.) and a SiH-content of 15.8 mmol/g was first dried by heating to 70° C. and a vacuum of less than 20 mbar for 3 hours. To this mixture was added 8.9 g LPNC (linear phosphoronitrile chloride) according to U.S. Pat. No. 4,203,913 as catalyst. The mixture was heated to 70° C. for 2 h, then 90° C. for 2 h, and finally held at 150° C. for 2 h while stirring. This gave an intermediate SiH-fluid of the general structure Me₃SiO(MeHSiO)₅₀(Me₂SiO)₁₅₂₀SiMe₃ and a viscosity of about 45.000 mPa*s at 25° C. (all measured with BOHLIN cone-plate-viscosimeter at shear rate of ω=6.28 sec⁻¹).

This intermediate was mixed with 166 g (0.99 mol) of an alpha olefin having molecular weight of 168 g/mol and the mixture was dried by heating to reflux at 130° C. and 40 mbar for 1 h using a water separator. After cooling to 25° C., 580 mg platinum in form of a Pt(0)-complex was added and the mixture was heated to 130° C. for 3 h. Afterwards the volatiles were removed by distillation at 150° C. and 1 mbar. The resulting product had an initial viscosity of 118,000 mPa*s at 25° C., a refractive index value of 1.4095 and contained 2.8% residual volatiles (volatiles means after 15 min 160° C. by thermogravimetry). The product has a brownish yellow opaque appearance. Volumetric determination of the residual SiH content found <0.01 mmol SiH/g. FTIR analysis found 15 ppm residual SiH in the product. After storage at room temperature in ambient air for 2 months the product had gelled.

### Example 2

### Intermediate Acc. to the Invention): M₂D^{R1}₅₀

A mixture of 11.25 g (0.18 mol SiH)SiH-siloxane having the general structure Me₃SiO(MeHSiO)₅₀SiMe₃ as in example 1 and 236 g alpha olefin having molecular weight of 168 g/mol was heated to 120° C., then 0.1 g Platinum in form of a Pt(0)-complex was added to catalyze the reaction. An additional 56.6 g (0.9 mol SiH) of the above SiH-polymer was added dropwise over 15 minutes. After 1 h at 130° C. 46 g of 1-hexene was added and mixture stirred for 2 hours. The reaction mixture was treated with 3 g activated charcoal and 1.5 g water and heated to 100° C. for 1 hour.

Then 3 g filteraid (diatomeceous earth) was added and the product filtered through a at Seitz K300 filter at 2 bar. The excess volatiles were removed at 150° C. and <20 mbar. The resulting product was a colorless, clear liquid with a viscosity of 743 mPa*s, and a residual volatiles content of <5%. Volumetric determination of the residual SiH content found <0.01 mmol SiH/g.

### Example 3

A mixture of 147 g of the reaction product of example 2 and 1430 g of octa-methylcyclotetrasiloxane were added to a stainless steel reactor. To the mixture was added 15.75 g of a 2% dispersion of CsOH in octamethylcyclotetrasiloxane (200 ppm CsOH) and dried by heating at reflux at 80° C. and a partial vacuum using a water separator. The reaction mixture was then heated to 180° C. for 6 hours to complete the equilibration. The product was neutralized with 2.3 g of a 3% solution of P₄O₁₀ in polydimethylsiloxane. The excess volatiles were removed for 1 hour at 150° C. and <1 mbar.

The resulting poly(methyl(C12-alkyl)siloxane-dimethylsiloxane) had a viscosity of 339,000 mPa·s, a refractive index of 1.4105 and 0.9% residual volatiles. FTIR analysis detected no residual SiH groups. After more than 1 year at room temperature the viscosity of the product had not changed.

### Example 4

Samples of 20 g of each of the poly(methyl(C12-alkyl)siloxane-dimethyl-siloxane) copolymers of example 1 and 3 are transferred into a beaker/bottle with a screw cap with an inner surface area of 25 cm² and stored under contact to ambient air at temperatures and times of tab.1.

TABLE 1

| Viscosity D = 1 s⁻¹ | | | | |
|---|---|---|---|---|
| Storage time after | | Example 1 | | |
| polymerization | | comparison | Ex. 3 | Ex. 5 |
| 4 h after equilibration | Pa · s | 118 | 330 | 72 |
| Color | | brownish- | colorless, | colorless, |
| | | yellow, opaque | clear | clear |
| 2 months )* | Pa · s | 489 | | |
| 1 year )* | Pa · s | gelled | 250 | 70 |

)* 25° C. 50% humidity ambient air

### Example 5

A mixture of 147 g of the reaction product of example 2 and 910 g of octa-methylcyclotetrasiloxane were added to a stainless steel reactor. To the mixture was added 10.6 g of a 2% dispersion of CsOH in octamethylcyclotetrasiloxane (200 ppm CsOH) and dried by heating at reflux at 80° C. and a partial vacuum using a water separator. The reaction mixture was then heated to 180° C. for 6 hours to complete the equilibration. The product was neutralized with 1.5 g of a 3% solution of P₄O₁₀ in polydimethylsiloxane. The excess volatiles were removed for 1 hour at 150° C. and <1 mbar.

The resulting poly(methyl(C12-alkyl)siloxane-dimethylsiloxane) had a viscosity of 71,600 mPa·s, a refractive index of 1.4105 and 2.3% residual volatiles. FTIR analysis detected no residual SiH groups. After more than 1 year at room temperature the viscosity of the product had not changed.

1. A process for the manufacture of polyorganosiloxanes comprising C6-C60 alkylmethylsiloxy-groups and dimethylsiloxy groups, the method comprising:
a) hydrosilylation of an C6-C60 olefin with a SiH-group-containing-polyorganosiloxane in the presence of a hydrosilylation catalyst,
b) subjecting the reaction product obtained in step a) to a reaction with at least one polydimethylsiloxane in the presence of a basic catalyst or phosphoronitrile chloride,
c) optionally neutralizing the catalyst used in step b),
d) optionally separating low volatiles from the reaction product obtained, or
e) hydrosilylation of an C6-C60-olefin with a SiH-group-containing-silane in the presence of a hydrosilylation catalyst,
f) subjecting the reaction product of step e) to polyorganosiloxane-formation,
g) subjecting the reaction product obtained in step f) to the reaction with at least one polydimethylsiloxane in the presence of a basic catalyst or phosphoronitrile chloride,
h) optionally neutralizing the catalyst used in step g), and
i) optionally separating low volatiles from the reaction product obtained.

2. The process of claim 1, wherein the C6-C60-olefin is selected from the group consisting of linear or branched aliphatic alpha-olefins, cyclic aliphatic olefins, fluoroalkyl-substituted olefins, and mixtures thereof.

3. The process of claim 1, wherein the C6-C60-olefin is selected from alpha-olefins selected from the group consisting of 1-hexene, 2-methyl-1-pentene, 3-methyl-1-pentene, 4-methyl-1-pentene, 1-heptene, 2-methyl-1-hexene, 1-octene, 2-methyl-1-heptene, 1-nonene, 1-decene, 1 undecene, 1-dodecene, 1-tridecene, 1-tetradecene, 1-pentadecene, 1-hexadecene, 1-octadecene, 1-nonadecene, 1-eicosene, hexacosene, octacosene, triacontene, tetratriacontene, hexatriacontene, octatriacontene, tetra-contene, dotetracontene, tetratetracontene, octa-tetracontene, pentacontene, dopentacontene, tetrapentacontene, hexa-pentacontene, octapentacontene and hexacontene, and cyclic olefins selected from cyclohexene, vinylcyclohexane, vinylcyclohexene, limonene, norbornene, ethylidene norbornene and dicyclopentadiene, and mixtures thereof.

4. The process of claim 1, wherein the SiH-group-containing-polyorganosiloxane has a SiH-group content of more than 50 mol-% based on the total amount of silicon atoms.

5. The process of claim 1, wherein the SiH-group-containing-polyorganosiloxane has a degree of polymerization of 1 to 200.

6. The process of claim 1, wherein the SiH-group-containing-polyorganosiloxane has the following formula:
(CH₃)ᵣHₛSi—O—[(CH₃)HSiO]₁₋₁₀₀Si(CH₃)ᵣHₛ
wherein r is 2 or 3, and s is 0 or 1.

7. The process of claim 1, wherein the SiH-group-containing-silane is selected from the group of methyldichlorohydrogensilane, dimethylchlorohydrogensilane, hydrogen(trialkoxy)silane, methylhydrogendialkoxysilane, and hydrogentrichlorosilane.

8. The process of claim 1, wherein the hydrosilylation catalyst is selected from one or more transition metals or transition metal compounds, wherein the transition metal is selected from the group consisting of platinum, rhodium, iridium, palladium, nickel and ruthenium, and mixtures thereof.

9. The process of claim 1, wherein the polydimethylsiloxane used in steps (b) or (g) is selected from cyclic, linear, or branched poly-dimethylsiloxanes.

10. The process of claim 1, wherein the polydimethylsiloxane is selected from cyclic polydimethylsiloxanes.

11. The process of claim 1, wherein the polydimethylsiloxane comprises a mixture comprising at least one cyclic polydimethylsiloxane and at least one trialkylsilyl-endcapped polydimethylsiloxane is used.

12. The process of claim 1, wherein the basic catalyst used in steps (b) or (g) is selected from the group consisting of alkaline metal hydroxides, ammonium hydroxides, phosphonium hydroxides and siloxanolates.

13. The process of claim 12, wherein the alkaline metal hydroxides used as catalyst in steps (b) or (g) are selected from potassium hydroxide, rubidium hydroxide, and cesium hydroxide, and wherein the ammonium and the phosphonium hydroxides are selected from tetraorganoammonium hydroxides and tetraorganophosphonium hydroxides, and wherein the siloxanolates are selected from potassium siloxanolates, rubidium siloxanolates, and cesium siloxanolates.

14. The process of claim 1, wherein steps (a) and (e) are carried out in a temperature range of 20 to 200° C.

15. The process of claim 1, wherein steps (b) and (g) are carried out in a temperature range of 80 to 180° C., and at a pressure of 1030 mbar.

16. The process of claim 1, wherein the polyorganosiloxanes manufactured by the process are triorgano-siloxy-endblocked polyorganosiloxanes.

17. Polyorganosiloxanes A polyorganosiloxane comprising (C6-C60)-alkylmethylsiloxy-groups and dimethylsiloxy groups obtained by the process of claim 1.

18. A polyorganosiloxane comprising (C6-C60)-alkylmethylsiloxy-groups and dimethylsiloxy groups of the formula
(CH₃)ᵣR¹ₛSi—O—[(CH₃)R¹SiO]_{y}[(CH₃)₂SiO]ₓSi(CH₃)ᵣR¹ₛ (IV′)
wherein r is 2 or 3,
s is 0 or 1,
R¹ is optionally substituted (C6-C60)-alkyl,
x>500, and
99>x/y>1.5.

19. The polyorganosiloxane of claim 18, wherein x+y is >600.

20. The polyorganosiloxane of claim 18, wherein x+y is >1000.

21. A process of adjusting the rheology of a composition comprising contacting polyorganosiloxane of claim 18 with the composition.

22. A process of conditioning the skin or hair comprising contacting the skin or hair with the polyorganosiloxane of claim 18.

23. A process of preventing dust comprising contacting the polyorganosiloxane of claim 18 with a composition that generates dust.

24. An aqueous emulsion comprising the polyorganosiloxane of claim 18.

25. An aqueous emulsion comprising the polyorganosiloxane of claim 19 and at least one emulsifier.
